(19) United States
(12) Patent Application Publication
(10) Pub. No.: US 2002/0115623 A1
(43) Pub. Date: Aug. 22, 2002

(54) Saponin derivatives useful for inhibiting sialyltransferase and biosynthesis of sialoglycoconjugate

(75) Inventors: Chi-Yue Wu, Taipei (TW); Shui-Tein Chen, Taipei (TW); Ying-Chieh Tsai, Taipei (TW)

Correspondence Address: Paul D. Greeley, Esq., Ohlandt, Greeley, Ruggiero & Perle, L.L.P., 10th Floor, One Landmark Square, Stamford, CT, 06901-2682, US

(73) Assignee: Shui-Tein Chen

(21) Appl. No.: 10/023,604

(22) Filed: Dec. 17, 2001

Related U.S. Application Data
(60) Provisional application No. 60/256,853, filed on Dec. 19, 2000.

(51) Int. Cl. A61K 31/704; C07H 15/24

(52) U.S. Cl. 514/33; 536/18.1

(57) ABSTRACT

The invention provides the use of the saponin derivatives, which is of the general formula (I) or the pharmaceutically acceptable salts and esters thereof:

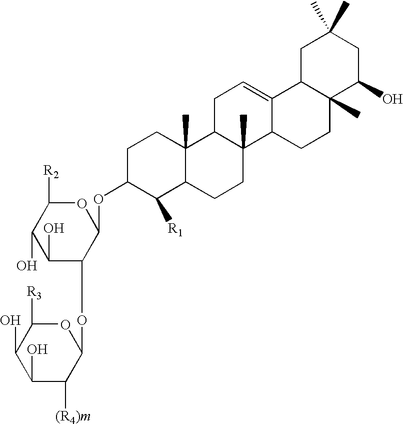

R₁ is hydrogen, C₁₋₈ alkyl, C₂₋₆ alkenyl, C₂₋₆ alkynyl, or C₁₋₈ alkylhydroxy;

R₂ is hydrogen, C₁₋₈ alkyl, C₂₋₆ alkenyl, C₂₋₆ alkynyl, COOH, COOC₁₋₈alkyl;

R₃ is C₁₋₈ alkylhydroxy, hydrogen, C₁₋₈ alkyl, C2-6 alkenyl or C₂₋₆ alkynyl;

R₄ is a sugar residue; and

m is 0, 1, 2 or 3;

and pharmaceutically acceptable carriers, as well as the use of such pharmaceutical composition in the inhibition of sialyltransferases.

### BACKGROUND OF THE INVENTION

1. Field of the Invention

The present invention relates to a new sialyltransferase inhibitor.

2. Description of the Prior Art

Sialyltransferase is a family of enzyme, which catalyzes the last step in the biosynthesis of complex oligosaccharides by transferring sialic acids onto terminal positions of carbohydrate group from glycoconjugates. It was known that the expression levels of sialyltransferases are significantly increasing during embryogenesis, growth, development, differentiation, immune defense, migration and homing of leukocytes, inflammation, allergy, infection by pathogens, oncogenic transformation, tumor metastatic potential and invasion (see Warren et. al., 1972, Proc Natl Acad Sci USA., 69(7), 1838-42; Oliver et. al., 1999, Glycobiology, 9(6), 557-569; Pilatte et al., 1993, 3(3), 201-18; and whitehouse et. al., 1997, J Cell Biol. 137(6), 1229-41).

Effective inhibitors of sialyltransferase and sialoglycoconjugate biosynthesis are thus expected to be useful as anti-inflammatory, immunosuppressive, anti-oncogenic, anti-metastatic, and invasive agents and therefore useful in the treatment of cancer and inflammatory.

The early development of the potent sialyltransferase inhibitors based on the structure of CMP-Neu5Ac has been described by Kleineidam et al., 1997, Glycoconjugate J, 14, 57-66. However, such inhibitors do not have good inhibition effect. They are not sufficient for the practically biological application. Further, the development of sialyltransferase inhibitors based on the transition state analogues of sialyl donor has been described by Schroder et al., Angew. Chem. Int. Ed., 1999, 38, No. 10, 1379-80. However, the developed inhibitors may not efficiently use in vivo to inhibit the sialyltransferases located in the intracellular Golgi apparatus. Up to now, no physiologically effective inhibitors of sialyltransferaseas are developed.

Saponin derivatives (saponins) are a series of the compounds isolated from solanaceous and leguminous plants. These compounds mainly include steroidal glycosides, treterpene glycosides, cholestane glycosides and cycloartane glycosides. Saponin derivatives are known in playing an important role in diseases treatment and physiological activities. For example, U.S. Pat. No. 5,591,836 discloses a saponin for use in lowering the cholesterol lever in blood. U.S. Pat. No. 5,968,516 provides a pharmaceutical composition containing saponins for treating cardiovascular disease, increasing immune function and decreasing lipids. Anti-tumor activities of the saponins are described in Takao Konoshima, Saponins Used in Traditional and Modem Medicine, Plenum Press, New York, 1996, pp. 87-100. Miyao et al., Planta medica 64 (1998) 5-7 discloses that the triterpene saponins have an antihepatotoxic activity.

However, none of the prior art discloses that saponins are useful as an effective sialyltransferase inhibitor and therefore useful in the treatment of the conditions associated with siayltransferase and sialoglycoconjugates such as cancer, inflammation, oncogenic transformation, metastasis and invasion.

### SUMMARY OF THE INVENTION

The object of the invention is to provide a saponin derivative useful for in inhibiting sialyltransferase activity, which is the general formula (I) or the pharmaceutically acceptable salts and esters thereof:

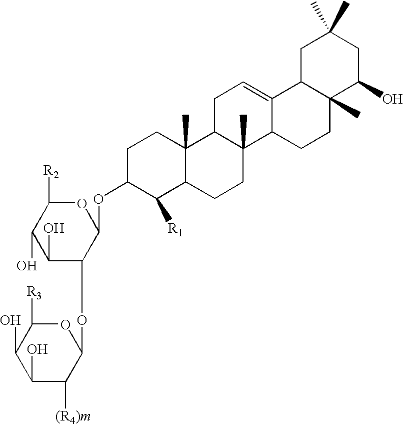

wherein

R₁ is hydrogen, C₁₋₈ alkyl, C₂₋₆ alkenyl, C₂₋₆ alkynyl, or C₁₋₈ alkylhydroxy;

R₂ is hydrogen, C₁₋₈ alkyl, C₂₋₆ alkenyl, C₂₋₆ alkynyl, COOH, COOC₁₋₈alkyl;

R₃ is C₁₋₈ alkylhydroxy, hydrogen, C₁₋₈ alkyl, C2-6 alkenyl or C₂₋₆ alkynyl;

R₄ is a sugar residue; and

m is 0, 1, 2 or 3.

### BRIEF DESCRIPTION OF THE DRAWINGS

FIG. 1 shows that Soyasaponin I specifically inhibits sialyltransferases but not for fucosyltransferase and galactosyltransferase.

FIG. 2 shows that soyasaponin I inhibits the growth of human breast cancer MCF7 cells.

FIG. 3 shows the inhibition of soyasaponin I on the growth of human hepatoma HepG2 cells.

### DETAILED DESCRIPTION OF THE INVENTION

The term “alkyl” as used herein refers to a substituted, straight or branched alkyl. Said alkyl includes, but is not limited to, methyl, ethyl, propyl, isopropyl, butyl, pentyl, hexyl and the like.

The term “alkenyl” as used herein refers to a substituted, straight or branched alkenyl. Said alkenyl includes, but is not limited to, ethenyl, propenyl, isopropenyl, butenyl, isobutenyl, hexenyl and the like.

The term “alkynyl” as used herein refers to a substituted, straight or branched alkynyl. Said alkynyl includes, but is not limited to, ethynyl, propynyl, butynyl, isobutnyl, hexynyl, and the like.

The term “alkylhydroxy” as used herein refers to a substituted, straight or branched alkylhydroxy. Said alkylhydroxy includes, but is not limited to, CH₂OH, CH₃CH₂OH, CH₃CH₂CH₂OH, and CH₃(CH₂)₃OH.

The term “sugar residue” as used herein refers to a residue of the sugar or the derivatives thereof. According to the invention, the sugar residue is preferably a pentose residue, hexose residue or derivatives thereof.

The term “pentose” as used herein refers to a monosaccharide containing five carbon atoms. Said pentose includes, but is not limited to, rhamnose, ribose, ribulose, lyxose, xylose, arabinose and xylulose. According to the invention, the pentose derivatives may be aldehydes, ketones and uronic acids of the pentose.

The term “hexose” as used herein refers to a monosaccharide containing six carbon atoms. Said hexose includes, but is not limited to, glucose, mannose, galactose and fructose. The hexose derivatives may be aldehydes, ketones and uronic acids of the hexose.

We have surprisingly found that the saponins of formula (I) provide a notable activity in the inhibition to the sialyltransferase and the biosynthesis of sialoglycoconjugates. By means of the inhibition of the sialyltransferase, the saponins according to the invention can be used as a therapeutic agent for the conditions associated with sialyltransferases and biosynthesis of sialoglycoconjugates.

One aspect of the invention is to provide a saponin derivative useful for inhibiting sialyltransferase, which is the general formula (I) or the pharmaceutically acceptable salts and esters thereof:

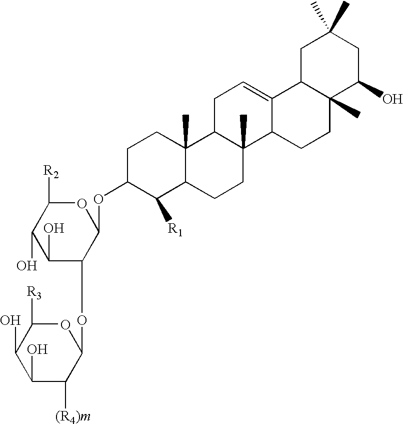

R₁ is hydrogen, C₁₋₈ alkyl, C₂₋₆ alkenyl, C₂₋₆ alkynyl, or C₁₋₈ alkylhydroxy;

R₂ is hydrogen, C₁₋₈ alkyl, C₂₋₆ alkenyl, C₂₋₆ alkynyl, COOH, COOC₁₋₈alkyl;

R₃ is C1-8 alkylhydroxy, hydrogen, C₁₋₈ alkyl, C2-6 alkenyl or C₂₋₆ alkynyl;

R₄ is a sugar residue; and

m is 0, 1, 2 or 3.

According to the invention, one preferred embodiment is the saponin of the formula (I) wherein R₁ is CH₂OH or CH₃; R₂ is COOH or COOMe; R₃ is CH₂OH or H; R₄ Rhamnose or galactose; and m is 0 or 1.

According to the invention, the more preferred embodiment is selected from soyasaponin I, soyasaponin II, kaikasaponin III, soyasaponin V and soyasaponin I-Methyl. The structures of the above-mentioned compounds are as follows:

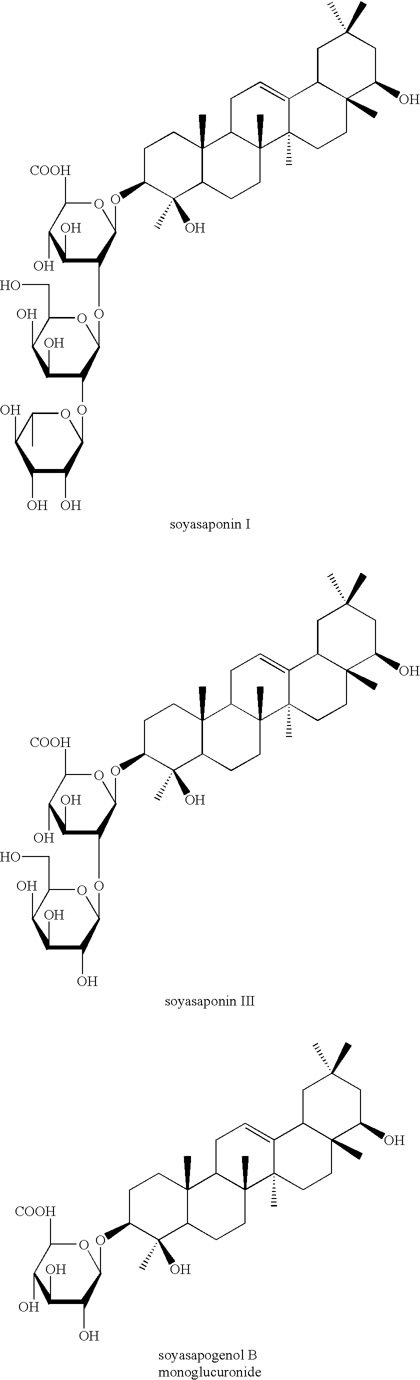

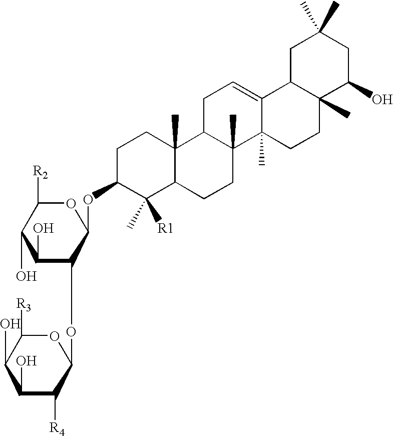

| | R1 | R2 | R3 | R₄ |
|---|---|---|---|---|
| soyasaponin I | CH₂OH | COOH | CH₂OH | Rha |
| kaikasaponin III | CH₃ | COOH | CH₂OH | Rha |
| soyasaponin I-Me | CH₂OH | COOMe | CH₂OH | Rha |
| soyasaponin II | CH₂OH | COOH | H | Rha |
| soyasaponin V | CH₂OH | COOH | CH₂OH | Glc |

According to the invention, the saponins can be extracted from solanaceous and leguminous plant by using the methods known in the art Ikeda et. al., 1998, Chem Pharm Bull (Tokyo), 46(2), 359-61; Miyao et. al., 1998, Planta Med, 64(1), 5-7.

It is found that saponins according to the invention show a high specific inhibition activity as to the sialyltransferase and the biosynthesis of sialoglycoconjugates. In particular, they are cell-impermeable molecules and exhibit the in vivo inhibition activity.

According to the invention, the saponins can be used in the treatment of the conditions associated with sialyltransferases and biosynthesis of sialoglycoconjugates. Preferably, the conditions include inflammation, allergy, infection by pathogens, oncogenesis, cancer, metastasis, and invasion caused by sialyltransferases. More preferably, the conditions are cancer, metastasis and invasion.

Another aspect of the invention is to provide a sialyltransferase inhibitor agent, which comprises a saponin derivative of formula (I).

Another aspect of the invention is to provide a method of inhibiting sialyltransferase, which comprises using the saponin derivative of formula (I).

The other aspect of the invention is to provide a method of treating the conditions associated with the sialyltransferase, which comprises administration of a sialyltransferase inhibitor agent of the invention to a patient suffering from, or susceptible to, such a condition. Preferably, the conditions include inflammation, allergy, infection by pathogens, oncogenesis, cancer, metastasis, and invasion caused by sialyltransferases. More preferably, the conditions are cancer, metastasis and invasion.

The amount of saponins in the sialyltransferase inhibitor agent of the invention will depend on the severity of the condition, and on the patient, to be treated, as well as the compounds which are employed.

Suitable doses of the sialyltransferase inhibitor agent according to the invention may be determined routinely by the medical practitioner or other skilled persons, and include the respective doses discussed in the prior art disclosing saponins that are mentioned hereinbefore, the disclosures in which are hereby incorporated by reference (see Wu et. al., 2001, Biochem Biophys Res Commun, 284(2), 466-9).

In any event, a physician, or a skilled person, will be able to determine the actual dosage which will be most suitable for an individual patient. The dosage is likely to vary with the condition that is to be treated, as well as the age, weight, sex and response of the particular patient to be treated.

The sialyltransferase inhibitor agent of the invention can be formulated for oral, rectal, paranteral or other mode of administration. The sialyltransferase inhibitor agent contains a saponin of the invention in combination with one or more pharmaceutically acceptable carrier. The carrier may be in the form of a solid, semi-solid or liquid diluent, or a capsule.

The following Examples are offered by way of illustration and not by way of limitation.

### EXAMPLES

### Example 1

The Preparation of Saponins

A. Preparation of Soyasaponin I and Kaikasaponin III

Abri Herba (5 kg) was purchased from local Chinese herbal company, Taiwan. Arbi Herba was extracted with MeOH (LC grade, Labscan) to isolate the active ingredients. The resulting extracts, after evaporation, were dissolved in 100% methanol and then filtered. The resulting filtrates were dissolved in a solution of 80% methanol and 20% H₂O. The n-hexane (UP grade, J. T. Beker) was added to the resulting solution. The methanol layer was collected, adjusted to 40% methanol with H₂O and then partitioned with ethylacetate (UP grade, J. T. Baker). The methanol layer was applied to a CHP-20P column (3 cm×35 cm, MCI Gel, Mitsubichi Chemical Industries LTD. Japan). Elution was carried out with a methanol linear gradient from 50% to 100% (total 4 liters). The fractions (from 85 to 95% methanol) containing soyasaponin I and kaikasaponin III were collected and concentrated under vacuum. Then, the sample was subjected to Nucleoside C₁₈ column (5 cm×25 cm) (Vydac). Elution was carried out with a methanol linear gradient from 60% to 100%. The fractions containing soyasaponin I and kaikasaponin III (from 80%-90% methanol) were combined and concentrated under reduced pressure. The resulting residues were then applied to a Nucleoside C₁₈ column (Vydac) (4.6 mm×250 mm) and separated using a linear gradient from 35% to 50% acetonitrile (Labscan), each containing 0.1% trifluoroacetic acid (TFA), for 30 min, at a flow rate of 1 ml/min. The partially purified soyasaponin I and kaikasaponin III were further purified by a BDS Phenyl column (Hypersil) (4.6 mm×250 mm) eluted with a linear gradient from 30% to 40% acetonitrile with 0.1% TFA at a flow rate of 1 ml/min. The samples were collected and lyophilized to obtain the white powder of soyasaponin I and kaikasaponin III, respectively.

B. Preparation of Soyasaponin II and Soyasaponin V

Crude soybean saponins (20 g) were purchased from Wako Pure Chem. Ind. Co. Ltd, Japan. The soyasaponin II and soyasaponin V were purified as described above. The white powder of soyasaponin II and soyasaponin V, respectively, were obtained after lyopholiaztion.

C. Preparation of Soyasaponin III

Soyasaponin I (10 mg) was dissolved in 3 ml of 0.5 N HCl-dioxane (1:1) (Merck) and heated at 100° C. for one hour. The reaction mixture was applied to a Nucleoside C18 column (Vydac) (4.6 mm×250 mm) eluted with a linear gradient from 35% to 100% acetonitrile, each containing 0.1% TFA, for 30 min at a flow rate of 1 ml/min. The fraction of soyasaponin III was collected and lyophilized to obtain the white powder of soyasaponin III.

D. Preparation of Soyasaponin I-Me

Soyasaponin I (5 mg) was dissolved in 1.5 ml of 0.01 N HCl-methanol and heated at 60° C. for one hour. The reaction mixture was applied to a Nucleoside C18 column (Vydac) (4.6 mm×250 mm) eluted with a linear gradient from 35% to 100% acetonitrile, each containing 0.1% TFA, for 30 min at a flow rate of 1 ml/min. The fraction of soyasaponin I-Me was collected and lyophilized to obtain the white powder of soyasaponin I-Me.

E. Mass Spectrometry

The mass spectra of Triterpenoid glycosides were recorded on an ion trap mass spectrometer (LCQ, Finnigan) equipped with an electrospray ionization source. The sample solution was introduced using a syringe pump. The mass spectrometer was operated in the positive mode, with electrospray ionization (ESI). The mobile phase was 50:50:1 acrtonitrile:water:acetic acid with a flow rate of 20 μl/minute.

| | Moleculare |
|---|---|
| | weight |
| Compound | [M + H] + (found) |
| soyasaponin I | 943.2 |
| soyasaponin II | 913.1 |
| kaikasaponin III | 927.4 |
| soyasaponin V | 959.4 |
| soyasaponin III | 797.3 |
| soyasaponin I-Me | 957.4 |

F. NMR Measurements

NMR spectra of the above-mentioned saponins were recorded on a BRUKER ARX500 spectrometer operating at 500.13 MHz and 125.76 MHz for 1H and 13C, respectively. The homonuclear experiments (DQF-COSY, 1H-1H-TOCSY, and NOESY) and the heteronuclear spectra (HMQC and HMBC) were also acquired.

### Example 2

Activity Assay

A. Assay of α2,3-sialyltransferase Activity

α2,3-Sialyltransferases were prepared by the procedures as described by Tsuji et al, Eur J Biochem., 1993, 216(2), 377-85. The suspension of enzyme-bound beads in 250 mM sodium cacodylate buffer (pH 6.4), 25 mM MgCl₂, 5 mM CaCl₂ was mixed with a Vortex mixer. Then, 4 μl of the enzyme suspension (1×10⁻⁶ Units) was pipetted using a Gilson P-20 pipetter immediately after mixing. The enzyme was then premixed with 4 μl of inhibitors (75 μM) or water (as control) at 25° C. for 5 min. Then, the substrates, 1 μl of CMP-[14C]NeuAc (40 nCi/μl) and 1 μl of Galα1,3GalNAc-Obzl (0.1 mM), were added to the pre-incubated enzyme-inhibitor mixture and incubated at 37° C. for 3 h. The final concentrations of sodium cacodylate, MgCl₂, CaCl₂, and Galα1,3GalNAc-OBzl in the reaction mixture were 100 mM, 10 mM, 2 mM, and 40 μM, respectively. The solution was placed in the ice to stop the reaction. The resulting reaction products were immediately analyzed by silica gel 60 HPTLC plate (Merck, Germany) with a solvent system of ethanol/pyridine/n-butanol/acetate/water (100:10:10:3:30). The radioactivity of the corresponding product and total radioactivity were detected and quantified with a BAS 1500 radio-image analyzer (Fuji Film, Japan).

The activity of the saponins of Example 1 in the inhibition of α2,3-sialyltransferaseon is shown in Table 1.

TABLE 1

| | Percentage of inhibition |
|---|---|
| Inhibitor* | (%) |
| Soyasaponin I | 89.9 |
| Soyasaponiin II | 93.3 |
| kaikasaponin III | 95.5 |
| Soyasaponin V | 79.6 |
| Soyasaponin III | 61.5 |
| Soyasaponin I-Me | 63.8 |

*The concentration of inhibitor in assay is 30 μM

B. Assay of α2,6-sialyltransferase Activity

α2,6-Sialyltransferases were prepared by the procedures as described by Tsuji et al, J Biol Chem., 1994, 269(2), 1402-9. The assay procedures for α2,6-Sialyltransferases were the same as those for α2,3-Sialyltransferases, except that 1 μl of Galα1,3GalNAc-Obzl (0.1 mM) was replaced by 1 μl of fetuin (1 μg/μl).

The activity of the saponins of Example 1 in the inhibition of α2,6-sialyltransferase is shown in Table 1.

TABLE 2

| | Percentage of inhibition |
|---|---|
| Inhibitor* | (%) |
| soyasaponin I | 76.9 |
| soyasaponin II | 64.4 |
| kaikasaponin III | 83.2 |
| soyasaponin V | 32.9 |
| soyasaponin III | 23.1 |
| soyasaponin I-Me | 32.2 |

*The concentration of inhibitor in assay is 20 μM.

### Example 3

Specificity Assay

Soyasaponin I was used to test the activity of various glycosidase.

A. β1.4-Galactosyltransferase Assay

The galactosyltransferase (1 mU/10 μl, Calbiochem) in 100 mM MOPS buffer (pH 7.4) and 40 mM MnCl₂ was mixed with 4 μl inhibitor or water, 4 μl N-acetylglucosamine (1 mM, Sigma) and 2 μl UDP-[¹⁴C]galactose (40 nCi,). The resulting solution was incubated at 37° C. for one hour. The enzyme reaction was performed within a period in which the reaction proceeded linearly. The reaction products were detected through the same procedures as that of sialyltransferase assay.

B. α(1¾) Fucosyltransferase Assay

The fucosyltransferase (1 mU/10 μl, Calbiochem) in 100 mM Tris-HCl buffer (pH 7.5) and 40 mM MnCl₂ was mixed with 4 μl inhibitor or water, 4 μl N-acetylglucosamine (1 mM) and 2 μl GDP-[¹⁴C]fucose (20 nCi). The resulting solution was incubated at 37° C. for one hour. The reaction products were detected through the same procedures as those for sialyltransferase assay.

C. Sialidase Assay

The sialidase (10 mU/90 μl, sigma) in 500 mM sodium phosphate buffer (pH 4.5) was mixed with 10 μl inhibitor (5 mM) or water, 100 μl 4-methylumbelliferyl-β-D-N-acetylneuraminic acid (0.2 mM, sigma). The resulting solution was incubated at 37° C. for 30 min. The solution was added 1 ml quenching buffer (0.133 M glycine-NaOH, pH 10.7, 60 mM NaCl and 41.5 mM Na₂CO₃) to stop the reaction. The fluorescence intensity of the resulting solution was measured by fluorescence spectrophotometer at 360 nm excitation and at 440 nm emission.

D. β-Galactosidase Assay

The β-galactosidase (10 mU/90 μl, Sigma) in 500 mM sodium phosphate buffer (pH 6.8) was mixed with 10 μl inhibitor (5 mM) or water, 100 μl 4-methylumbelliferyl-β-D-galactopyranoside (0.2 mM). The resulting solution was incubated at 37° C. for 30 min. The resulting products were detected through the same procedures as those for sialidase assay.

E. α-Mannosidase Assay

The α-mannosidase (10 mU/90 μl, Sigma) in 500 mM sodium phosphate buffer (pH 4.5) was mixed with 10 μl inhibitor (5 mM) or water, 100 μl 4-methylumbelliferyl-β-D-mannopyranoside (0.2 mM). The resulting solution was incubated at 37° C. for 30 min. The resulting products were detected through the same procedures as those for sialidase assay.

F. Results

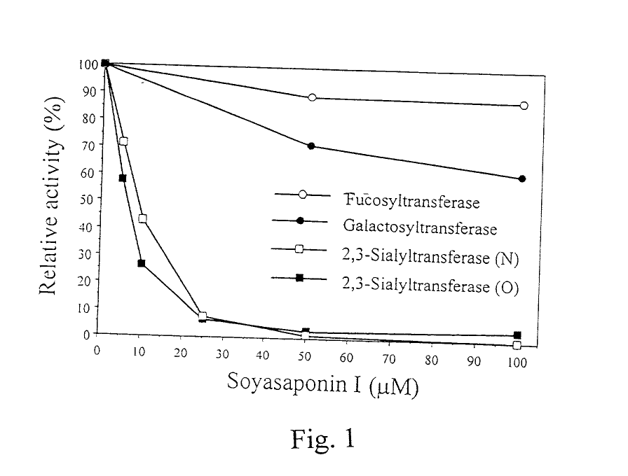

The results of the above assays are shown in Table 3 and FIG. 1. As shown in Table 3, soyasaponin I did not inhibit galactosidase, mannosidase and sialidase. In addition, as shown in FIG. 1, soyasaponin I specifically inhibited sialyltransferases but not for fucosyltransferase and galactosyltransferase. Given the above, the saponins of the invention have a specific inhibition in the sialyltransferases.

TABLE 3

| | | |
|---|---|---|
| | | Relative Activity (%) |
| | Relative Activity (%) | (soyasaponin I, 250 |
| Glycosidase | (Control) | mM) |

| | | |
|---|---|---|
| B-galactoside | 100 | 112.0 |
| A-mannosidase | 100 | 99.4 |
| Sialidase | 100 | 142.0 |

### Example 4

Cell Assay

A. Culture of Human Breast Cancer Cell MCF7 and Hepatoma Cell HepG2

Human breast cancer cell MCF7 and hepatoma cell HepG2 were incubated in 5% CO₂ incubator at 37° C. with the MEM (Eagle) medium with Earle's Bss supplemented with 2 mM glutamine, 1.5 g/l sodium bicarbonate, 0.1 mM non-essential amino acids, 1.0 mM sodium pyruvate, 100 units/ml penicillin, 100 μg/ml streptomycin and 2.5 μg/ml amphotericin B and with 10% fetal bovine serum. The cells were transferred weekly to 0.025% trypsin in 0.53 mM EDTA in Ca²⁺Mg²⁺-free PBS.

B. Inhibition of MCF7 and HepG2 Cells

The cells were incubated in the culture medium with 2.5% fetal bovine serum in the presence of a different concentration (5, 25, 50 and 100 μM, respectively) of the inhibitor in a 5% CO₂ incubator at 37° C. for three days.

C. Flow Cytometry of Inhibitor-treated Cells

Fluorescein isothiocyanate labeled Maackia amurensis agglutinin (MAA) and Sambucus nigra agglutinin (SNA), which recognize the oligosaccharide species NeuAcα2,3-Gal-R and NeuAcα2,6-R, respectively, were purchased from Vector Labs Inc. (Burlingame, Calif.). Cells (control and inhibitor-treated). The cells scraped from dishes with a rubber policeman were washed with PBS and stained with fluorescein isothiocyanate labeled MAA and SNA, respectively, in binding buffer (10 mM HEPES, 0.15 M NaCl, pH 7.5) at 4° C. for 1 h. After centrifugation, cells were washed three times with PBS and fixed with 1% paraformaldehyde. The fluorescence intensity was measured with a FACScan flow cytometry (Becton Dickinson, Mountain View, Calif.).

D. Results

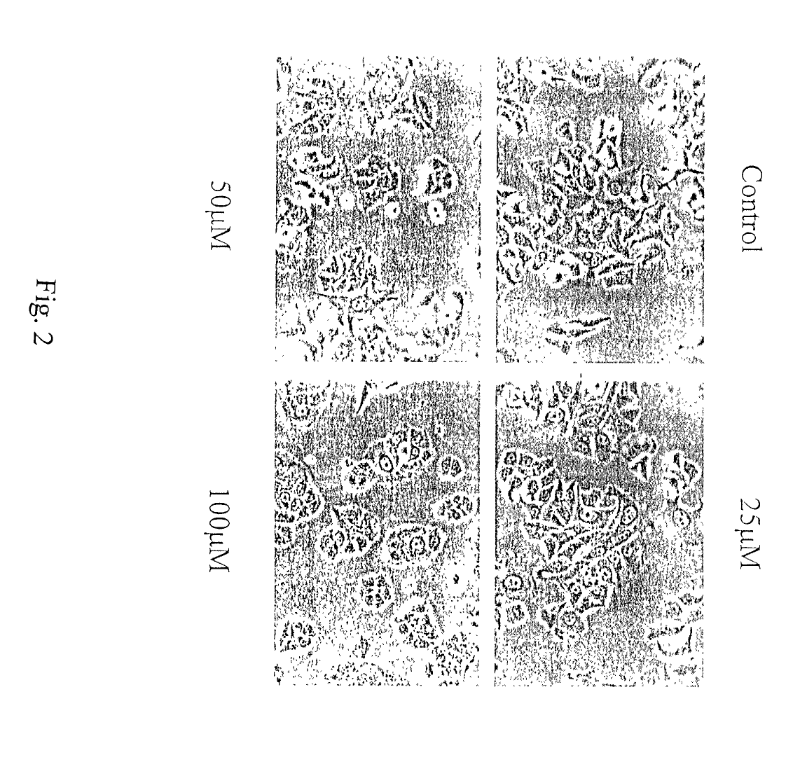

As shown in Table 4, soyasaponin I inhibited the expression of cell surface α2,3-sialoglyconjugates of human breast cancer MCF7 cells. 100 μM of soyasaponin I can decrease about 80% of the α2,3-sialoglyconjugates of MCF7 cells. Moreover, as shown in FIG. 2, soyasaponin I inhibited the growth of human breast cancer MCF7 cells.

TABLE 4

| | Cell surface |
|---|---|
| Inhibitors (μM) | α2,3-sialoglycoconjugates (%) on MCF7* |
| Control | 100 |
| soyasaponin I (5) | 99.5 |
| soyasaponin I (25) | 68.0 |
| soyasaponin I (50) | 56.8 |
| soyasaponin I (100) | 19.8 |

*The quantity of cell surface α2,3-sialoglyconjugates was measured by FITC-MAA stain and FACScan cytometry.

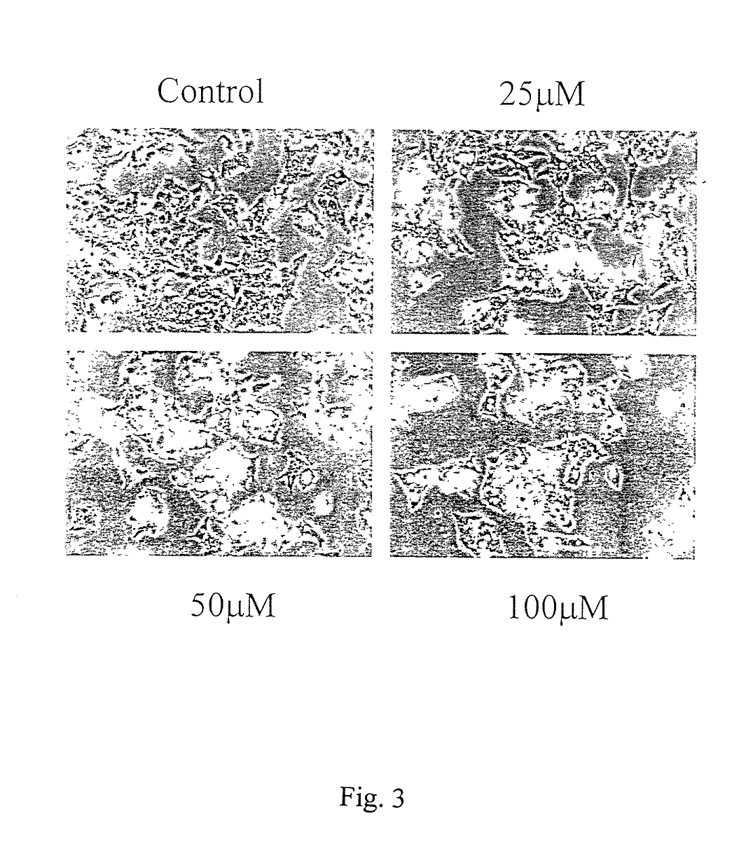

In addition, soyasaponin I inhibited the expression of cell surface α2,6-sialoglyconjugates of human hepatoma HepG2 cells. As shown in Table 5, 100 μM of soyasaponin I decreased about 60% cell surface α2,6-sialoglyconjugates of HepG2 cells. Also, as shown in FIG. 3, the soyasaponin I inhibited the growth of human hepatoma HepG2 cells.

TABLE 5

| | Cell surface |
|---|---|
| | α2,6-sialoglycoconjugates |
| Inhibitors (μM) | (%) on HepG2* |
| Control | 100 |
| soyasaponin I (5) | 96.5 |
| soyasaponin I (25) | 85.1 |
| soyasaponin I (50) | 76.4 |
| soyasaponin I (100) | 41.4 |

*The quantity of cell surface α2,6-sialoglyconjugates was measured by FITC-SNA stain and FACScan flow cytometry

What is claimed is:

1. A saponin derivative useful for inhibiting sialyltransferase, which is the general formula (I) or the pharmaceutically acceptable salts and esters thereof: 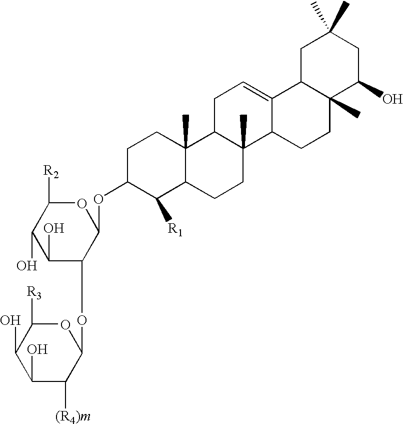
wherein
R₁ is hydrogen, C₁₋₈ alkyl, C₂₋₆ alkenyl, C₂₋₆ alkynyl, or C₁₋₈ alkylhydroxy;
R₂ is hydrogen, C₁₋₈ alkyl, C₂₋₆ alkenyl, C₂₋₆ alkynyl, COOH, COOC₁₋₈alkyl;
R₃ is C₁₋₈ alkylhydroxy, hydrogen, C₁₋₈ alkyl, C2-6 alkenyl or C₂₋₆ alkynyl;
R₄ is pentose or hexose residue or their derivatives; and
m is 0, 1, 2 or 3.

2. The saponin derivative of claim 1, wherein R₁ is CH₂OH or CH₃; R₂ is COOH or COOMe; R₃ is CH₂OH or H; and R₄ is rhamnose or galactose.

3. The saponin derivative of claim 1, wherein the saponin of formula (I) is selected from soyasaponin I, soyasaponin II, kaikasaponin III, soyasaponin V and soyasaponin I-Methyl.

4. The saponin derivative of claim 1, which can be used in the treatment of the disease associated with sialyltransferase.

5. The saponin derivative of claim 4, wherein the diseases is selected from inflammation, allergy, infection by pathogens, oncogenesis, cancer, metastasis and invasion.

6. The saponin derivative of claim 5, wherein the disease is cancer, metastasis or invasion.

7. A sialyltransferase inhibitor agent, which comprises the saponin derivative as defined in claim 1.

8. The agent of claim 7, wherein R₁ is CH₂OH or CH₃; R₂ is COOH or COOMe; R₃ is CH₂OH or H; and R₄ is rhamnose or galactose.

9. The agent of claim 7, wherein the saponin of formula (I) is selected from soyasaponin I, soyasaponin II, kaikasaponin III, soyasaponin V and soyasaponin I-Methyl.

10. A method of inhibiting sialyltransferase, which comprises using the saponin derivative as defined in claim 1.

11. A method of treating the conditions associated with the sialyltransferase, which comprises administration of a sialyltransferase inhibitor agent of the invention to a patient suffering from, or susceptible to, such a condition.

12. The method of claim 11, wherein the condition is selected from inflammation, allergy, infection by pathogens, oncogenesis, cancer, metastasis, and invasion caused by sialyltransferases.

13 The method of claim 12, wherein the condition is selected from cancer, metastasis and invasion.
